(19) United States
(12) Patent Application Publication
(10) Pub. No.: US 2025/0191714 A1
(43) Pub. Date: Jun. 12, 2025

(54) SYSTEM AND METHOD FOR GENERATING DISEASE ANALYSIS REPORT

(71) Applicant: INTOWELL BIOMEDICAL TECHNOLOGY, INC., Taipei City (TW)

(72) Inventor: Chien-Chung LIAO, Hsinchu (TW)

(73) Assignee: INTOWELL BIOMEDICAL TECHNOLOGY, INC., Taipei City (TW)

(21) Appl. No.: 18/972,673

(22) Filed: Dec. 6, 2024

Related U.S. Application Data
(60) Provisional application No. 63/607,020, filed on Dec. 6, 2023.

(51) Int. Cl. G16H 15/00; G06F 40/205; G16H 10/60

(52) U.S. Cl. CPC G16H 15/00; G06F 40/205; G16H 10/60

(57) ABSTRACT

A method and system for generating medical analysis reports are proposed. The method includes: receiving a piece of text data, extracting at least one piece of first sub-data using a parsing module according to a natural language processing technique, analyzing the first sub-data using at least one medical database and at least one rule engine to generate at least one first intermediate result, executing a large language model to generate a first medical analysis report according to the at least one first intermediate result, using the parsing module and feedback information to parse the first medical analysis report or the piece of text data into at least one piece of second sub-data, which is analyzed to generate at least one second intermediate result, and a second medical analysis report is generated according to the at least one second intermediate result.

### CROSS-REFERENCE TO RELATED APPLICATIONS

This non-provisional application claims priority under 35 U.S.C. § 119(a) on Patent Application No(s). 63/607,020 filed in U.S. on Dec. 6, 2023, the entire contents of which are hereby incorporated by reference.

### BACKGROUND

### 1. Technical Field

The present disclosure relates to data analysis, specifically to a system and method for generating disease/medical analysis reports.

### 2. Related Art

When a patient is admitted to the hospital, healthcare providers are required to manually prepare a series of critical documents, including an admission note (AN), an admission order (AO), and a progress note (PN). These documents are essential for the treatment and management of patients, but healthcare providers often face multiple challenges when generating them.

First, preparing these documents requires a significant amount of time and effort. Healthcare providers must meticulously record the patient's medical history, physical examination results, diagnosis, and treatment plans, making this a tedious and time-consuming process. Second, this documentation work often prevents healthcare providers from dedicating more time to direct patient care. Moreover, these documents require frequent revisions. As the patient's condition changes, healthcare providers must continuously update the progress notes to reflect the latest diagnosis and treatment plans. This forces healthcare providers to return repeatedly to the documentation process, further increasing their workload.

### SUMMARY

In light of the above descriptions, the present disclosure proposes a system and method for generating medical analysis reports to address the aforementioned issues.

According to one or more embodiment of the present disclosure, a method for generating medical analysis reports is performed by a computing device, and includes the following steps: receiving a piece of text data; extracting at least one piece of first sub-data from the piece of text data using a parsing module according to a natural language processing technique; analyzing the at least one piece of first sub-data using at least one medical database and at least one rule engine to generate at least one first intermediate result; executing a large language model to generate and output a first medical analysis report according to the at least one first intermediate result; using the parsing module and feedback information in response to the first medical analysis report to parse the first medical analysis report or the piece of text data into at least one piece of second sub-data; analyzing the at least one piece of second sub-data using the at least one medical database and the at least one rule engine to generate at least one second intermediate result; and executing the large language model to generate and output a second medical analysis report according to the at least one second intermediate result.

According to one or more embodiment of the present disclosure, a system for generating medical analysis reports includes an input device, a storage device, a computing device and an output device. The input device is configured to receive a piece of text data, a first medical analysis report, and feedback information. The storage device is configured to store a parsing module, at least one medical database, at least one rule engine, and a large language model. The computing device is communicably connected to the input device and the storage device. The computing device is configured to extract at least one piece of first sub-data from the piece of text data using the parsing module according to a natural language processing technique; analyze the at least one piece of first sub-data using the at least one medical database and the at least one rule engine to generate at least one first intermediate result; execute the large language model to generate and output the first medical analysis report according to the at least one first intermediate result; use the parsing module and the feedback information in response to the first medical analysis report to parse the first medical analysis report or the piece of text data into at least one piece of second sub-data; analyze the at least one piece of second sub-data using the at least one medical database and the at least one rule engine to generate at least one second intermediate result; executing the large language model to generate and output a second medical analysis report according to the at least one second intermediate result. The output device communicably connected to the computing device to output the first medical analysis report and the second medical analysis report.

In view of the above, the system and method for generating medical analysis reports proposed in the present disclosure possess advanced data analysis capabilities, excelling in interpreting complex medical records, akin to a general practitioner accurately summarizing and sequentially analyzing symptoms. Furthermore, the proposed system and method include quality control mechanisms to identify potential issues within medical reports, reducing human errors and omissions, thereby ensuring the highest quality of patient care information. Additionally, the system and method enable real-time support for medical care, automating routine medical documentation tasks, and allowing teams to share consistent and up-to-date patient records.

### BRIEF DESCRIPTION OF THE DRAWINGS

The present disclosure will become more fully understood from the detailed description given hereinbelow and the accompanying drawings which are given by way of illustration only and thus are not limitative of the present disclosure and wherein:

FIG. 1 is a block diagram of a system for generating medical analysis reports according to an embodiment of the present disclosure;

FIG. 2 is a data flow diagram of a plurality of operations of a software system running on a computing device;

FIG. 3 is an internal structural diagram of the analysis module; and

FIG. 4 is a flowchart of a method for generating medical analysis reports according to an embodiment of the present disclosure.

### DETAILED DESCRIPTION

In the following detailed description, for purposes of explanation, numerous specific details are set forth in order to provide a thorough understanding of the disclosed embodiments. According to the description, claims and the drawings disclosed in the specification, one skilled in the art may easily understand the concepts and features of the present invention. The following embodiments further illustrate various aspects of the present invention, but are not meant to limit the scope of the present invention.

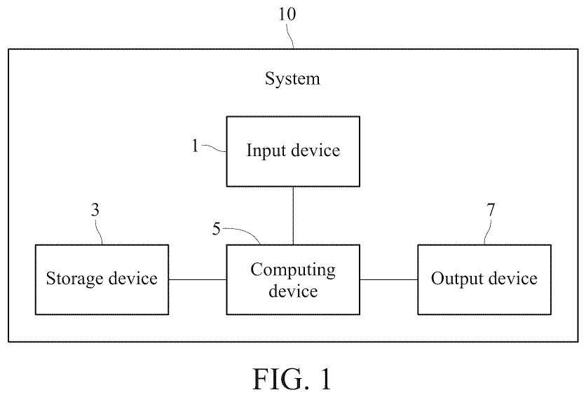

FIG. 1 is a block diagram of a system for generating medical analysis reports according to an embodiment of the present disclosure. As shown in FIG. 1, the system 10 for generating medical analysis reports includes an input device 1, a storage device 3, a computing device 5, and an output device 7.

The input device 1 is configured to receive a piece of text data, a first medical analysis report, and feedback information. In an embodiment, the input device 1 may be a hardware component such as a keyboard, mouse, or touchpad. In another embodiment, the input device 1 may be a software component such as an Application Programming Interface (API) or a database. However, the present disclosure is not limited to these examples. The source of the text data can be direct user input or text obtained through voice or image conversion.

The storage device 3 is configured to store a parsing module 50, at least one medical database, at least one rule engine, and a large language model (LLM). In an embodiment, the storage device 3 may be, for example, flash memory, a hard disk drive (HDD), a solid-state drive (SSD), dynamic random-access memory (DRAM), static random-access memory (SRAM), or other non-volatile memory. However, the present disclosure is not limited to these examples.

The computing device 5 is communicably connected to the input device 1 and the storage device 3. The computing device 5 is configured to run the software system proposed by the present disclosure, the eXpertMind™ Engine. This software system includes the following multiple operations:
extracting at least one piece of first sub-data from the piece of text data using the parsing module 50 according to a natural language processing technique; analyzing the at least one piece of first sub-data using the at least one medical database and the at least one rule engine to generate at least one first intermediate result; executing a large language model to generate and output the first medical analysis report according to the at least one first intermediate result; using the parsing module 50 and the feedback information in response to the first medical analysis report to parse the first medical analysis report or the piece of text data into at least one piece of second sub-data; analyzing the at least one piece of second sub-data using the at least one medical database and the at least one rule engine to generate at least one second intermediate result; executing the large language model to generate and output a second medical analysis report according to the at least one second intermediate result. In an embodiment, the computing device 5 may adopt at least one of the following examples: a personal computer, a network server, a central processing unit (CPU), a graphics processing unit (GPU), a neural network processing unit (NPU), a microcontroller (MCU), an application processor (AP), a field-programmable gate array (FPGA), an application-specific integrated circuit (ASIC), a system-on-a-chip (SOC), a deep learning accelerator, or any other electronic device with similar functionalities. The present disclosure does not limit the hardware type of the computing device 5.

The output device 7 is communicably connected to the computing device 5 to output the first medical analysis report and the second medical analysis report. In an embodiment, the output device 7 may be any hardware or software component that provides graphical and textual outputs. Examples of hardware components include screens, projectors, and speakers, while examples of software components include API interfaces or databases. The present disclosure does not limit the type of output device 7.

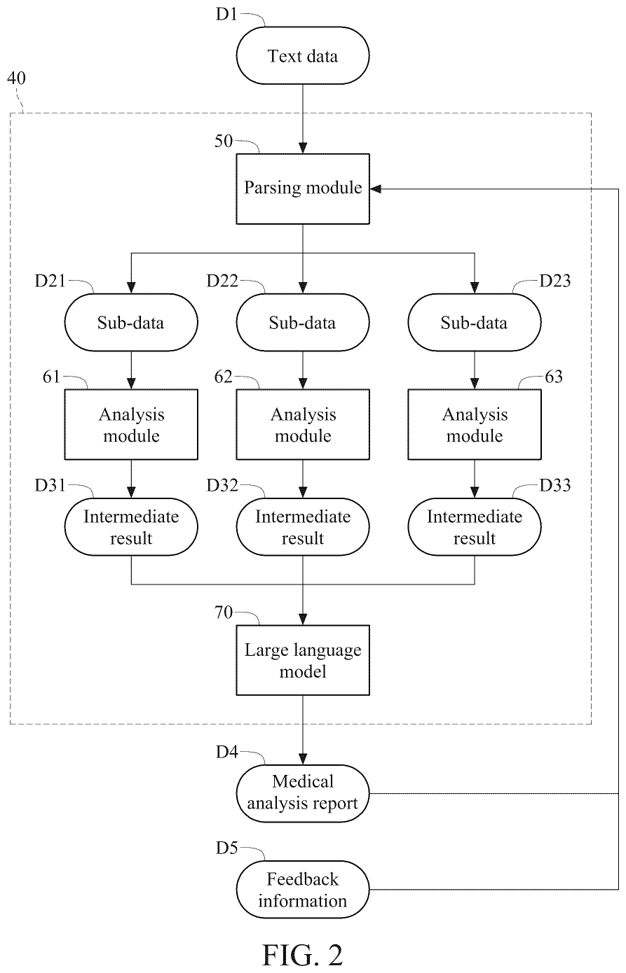

FIG. 2 is a data flow diagram of a plurality of operations of a software system running on a computing device. As shown in FIG. 2, the software system 40 includes a parsing module 50, analysis modules 61, 62, and 63, and a large language model 70. A notable feature of the present disclosure is the ability to re-generate medical analysis reports. In other words, if the user is dissatisfied with the first-round medical analysis report D4, it can be returned to the software system 40, which then generates a new medical analysis report based on the previous report D4 and feedback information D5.

The first-round operation process of the software system 40 is as follows: A user generates a piece of text data DI via the input device 1. The parsing module 50 parses the piece of text data DI into a plurality pieces of sub-data D21, D22, and D23. The analysis modules 61, 62, and 63 utilize the built-in medical database and rule engine to analyze the plurality pieces of sub-data D21, D22, and D23, and to generate a plurality of intermediate results, D31, D32, and D33. The large language model 70 generates the medical analysis report D4 according to the plurality of intermediate results, D31, D32, and D33. The second-round operation process of the software system 40 is as follows: The medical analysis report D4 generated in the first round is re-input into the software system 40. The user may optionally provide feedback information D5 according to the medical analysis report D4. The parsing module 50 then parses the feedback information D5 and/or the piece of text data DI into a plurality of new pieces of sub-data. The analysis modules 61, 62, and 63 analyze these new pieces of sub-data, and generates new intermediate results. Finally, the large language model 70 generates a new medical analysis report according to the new intermediate results.

The piece of text data DI may be, for example, an admission note drafted by professionals, with content that may include, but is not limited to, chief complaint, present illness, review of systems, physical exam, and clinical laboratory results. The first medical analysis report D4, for example, may be an original analysis (OA). The feedback information D5 includes user ratings and prompts regarding the medical analysis report D4. The ratings are used to adjust the analysis process of the software system 40 to improve the quality of the newly generated medical analysis reports in subsequent rounds. The software system 40 can record the types of user queries, query content, and feedback information D5.

A high score indicates user's high satisfaction with the suggestions generated by the software system 40, leading to increased weight for the current analysis strategy for similar issues in the future. A low score indicates that the results did not meet the user's expectations or requirements, leading the system to reduce the weight of the current analysis strategy for similar issues in the future and attempt different parameter settings to adjust the analysis strategies of the analysis modules 61, 62, and 63, thereby generating more appropriate results. Prompts are user commands to instruct the software system 40 to generate a new format of the medical analysis report D4 or retain the existing format of the medical analysis report D4. After obtaining OA₁, the user may use prompts to command the software system 40 to repeatedly generate new reports, such as OA₂, OA₃, . . . , OA_{N}. The user may also use prompts to command the software system 40 to generate other formats of the medical analysis report D4, such as Admission Notes (AN), Admission Orders (AO), or Progress Notes (PN). Furthermore, the process of generating new formats of the medical analysis report D4 can be repeated. For example, after generating AN₁/AO₁/PN₁, if the user is dissatisfied with their content, new versions such as AN₂/AO₂/PN₂ can be generated in the same manner as described above.

The parsing module 50 adopts natural language processing (NLP) technique to understand and analyze the user's input. NLP technique can identify keywords, semantic structures, and intent.

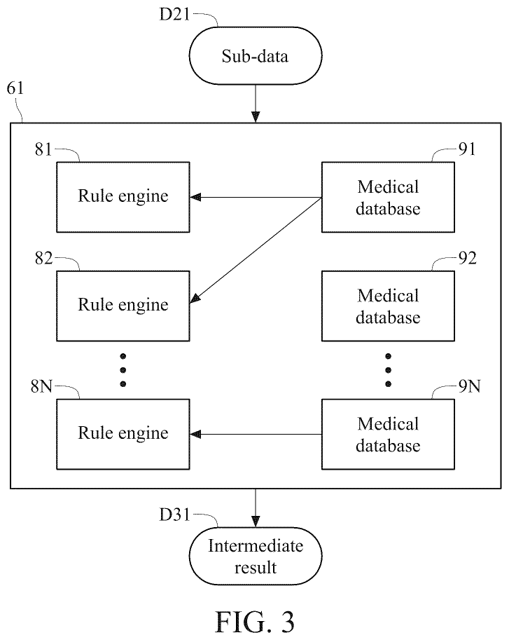

FIG. 3 is an internal structural diagram of the analysis module (taking 61 as an example). As shown in FIG. 3, the analysis module 61 includes a plurality of rule engines 81, 82 . . . 8N and a plurality of medical databases 91, 92 . . . 9N. The rule engines 81, 82 . . . 8N use the keywords, semantic structures, and data results identified by the parsing module 50, combined with the vast amounts of medical data and expert knowledge stored in the medical databases 91, 92 . . . 9N, and adjust the weight of the analysis strategy according to different feedback information D5. The configuration of the analysis module described above operates as an expert system that simulates the thought logic of a professional doctor to perform corresponding analyses on multiple items. These items include, but are not limited to: differential diagnosis, information requiring further understanding, most likely diagnosis, host factors, speculated pathogenic mechanisms, other diagnostic tests, imaging exams, urgent treatment required, medication treatment, potential complications, preventive measures after management, etc.

In an embodiment, the large language model 70 and medical databases 91, 92 . . . 9N generate a piece of reference data as a part of the intermediate results D31, D32, and/or D33 based on a retrieval-augmented generation (RAG) technique.

Tables 1, 2, and 3 below are used to illustrate specific examples of the pieces of text data DI, sub-data D21, D22, and D23, and intermediate results D31, D32, and D33 shown in FIG. 2 and FIG. 3. where Table 3 is additionally used to present the process of rule engine operation. However, the present disclosure is not limited to these examples.

TABLE 1
example of the piece of text data D1.

The individual is a 49-year-old male born on Oct. 10, 1967. He has a
medical history that includes diabetes, hyperlipidemia (high cholesterol),
hypertension (high blood pressure), and is a carrier of hepatitis B. Recent
laboratory examinations showed the following results: GOT at 190 U/L,
GPT at 149 U/L, albumin (ALB) at 39 g/L, globulin (GLOB) at 40 g/L,
γ-GT at 309 U/L, direct bilirubin (D-BIL) at 20.52 umol/L, indirect
bilirubin (I-BIL) at 20.52 umol/L, total bilirubin (T.BIL) at 41.04 umol/L,
alkaline phosphatase (ALK-P) at 668 U/L, IgG anti-HAV at 2.20 (+)
S/CO, HBsAg at 7411 (+) Index, and anti-HCV at 0.1 (−) Index.

TABLE 2
example of sub-data D21.

| Type | Item | Value | | Note |
|---|---|---|---|---|
| Basic Data | Gender | Male | | |
| Basic Data | DoB | Oct. 10, 1937 | | |
| Basic Data | Age | 49 | | |
| Personal History | diabetes | Positive | | |
| Personal History | hyperlipidemia | Positive | | |
| Personal History | hypertension | Positive | | |
| Personal History | hepatitis B | Positive | | Carrier |
| Lab Exam | GOT | 190 | | U/L |
| Lab Exam | GPT | 149 | | U/L |
| Lab Exam | ALB | 39 | | g/L |
| Lab Exam | GLOB | 40 | | g/L |
| Lab Exam | γ-GT | 309 | | U/L |
| Lab Exam | D-BIL | 20.52 | | umol/L |
| Lab Exam | I-BIL | 20.52 | | umol/L |
| Lab Exam | T.BIL | 41.04 | | umol/L |
| Lab Exam | ALK-P | 668 | | U/L |
| Lab Exam | IgG anti-HAV | 2.20 | (+) | S/CO |
| Lab Exam | HBsAg | 7411 | (+) | Index |
| Lab Exam | Anti-HCV | 0.1 | (−) | Index |

TABLE 3
example of the operation of the rule engine and medical database.

| | |
|---|---|
| Condition | Rule or Result |

Step. 1 - Finding (Ruleset 81 from Medical Database 91)

| | |
|---|---|
| | GOT >40 |
| and | |
| | GPT >40 |

Step. 2 - Finding (Ruleset 82 from Medical Database 91)

| | |
|---|---|
| | HBsAg Positive |
| and | |
| | hepatitis B Carrier |

Step. 3 - Finding (Ruleset 83 from Medical Database 91)

| | |
|---|---|
| | Total Bilirubin >=2 |
| and | |
| | Indirect Bilirubin/Total Bilirubin <0.6 |
| and | |
| | Direct Bilirubin/Total Bilirubin >0.4 |

Step. 4 - Result (Diagnosis 84 from Medical Database 92)

Chronic hepatitis B with decompensation

Step. 5 - Suggestion (Suggestion 85 from Medical Database 93)

Please return to the department of gastroenterology
immediately to receive medicinal treatments (e.g. anti-viral
drugs) and abdominal ultrasound. Please return for follow-up
blood tests in three months.

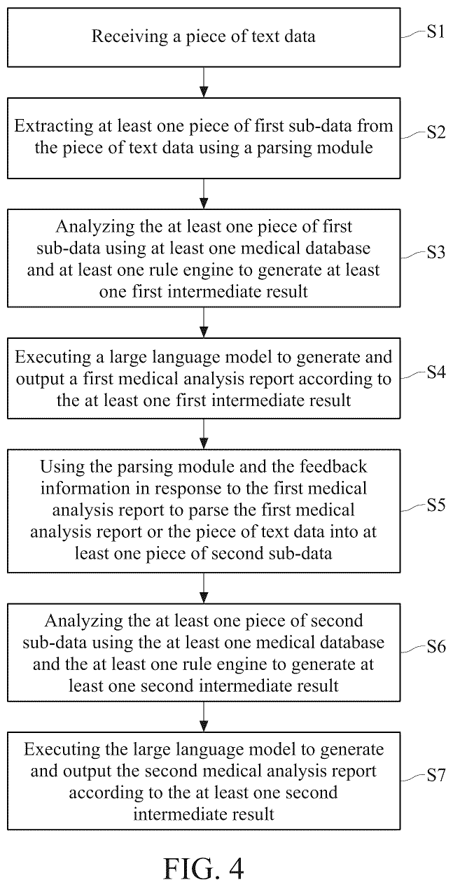

FIG. 4 is a flowchart of a method for generating medical analysis reports according to an embodiment of the present disclosure, including steps S1 to S7. In step S1, the computing device 5 receives the piece of text data from the input device 1. In step S2, the computing device 5 extracts at least one piece of first sub-data from the piece of text data using a parsing module 50 according to a natural language processing technique. In step S3, the computing device 5 analyzes at least one piece of first sub-data using at least one medical database and at least one rule engine to generate at least one first intermediate result. In step S4, the computing device 5 executes the large language model 70 to generate and output the first medical analysis report according to the at least one first intermediate result. In step S5, the computing device 5 uses the parsing module 50 and the feedback information in response to the first medical analysis report to parse the first medical analysis report or the piece of text data into at least one piece of second sub-data. In step S6, the parsing module 50 analyzes the at least one piece of second sub-data using at least one medical database and at least one rule engine to generate at least one second intermediate result, this step includes adjusting the weight used in the analysis according to the feedback information to generate the at least one second intermediate result, where the weight corresponds to an acceptability of the feedback information, and the at least one second intermediate result is different from the at least one first intermediate result. In step S7, the computing device 5 executes the large language model 70 to generate and output the second medical analysis report according to the at least one second intermediate result. In the above process, it may further include: the computing device 5 generates a piece of reference data using the large language model 70 and the at least one medical database based on a retrieval-augmented generation technique, where the piece of reference data serves as a part of the at least one first intermediate result.

The following describes an application scenario of the system and method for generating medical analysis reports proposed by the present disclosure. First, a healthcare professional provides a piece of text data to the system 10 for generating medical analysis reports, as proposed in an embodiment of the present disclosure. Then, the software system 40 running on the computing device 5 performs an initial analysis according to the piece of text data, comprehensively evaluating the patient's clinical condition and generating the first original analysis (OA₁). If the result of OA₁ is unacceptable, feedback information and a prompt can be entered, and OA₁ along with the feedback information are submitted to the software system 40. The software system 40 then generates a new OA₂ according to the piece of text data and the previous OA₁. This process may be repeated as needed, generating OA₃, OA₄, . . . , OA_{X}. Next, the healthcare professional selects the most accurate analysis from these original analyses, for example, OAᵢ, and re-enters it into the software system 40 along with the prompt and the piece of text data. The prompt requests the provision of the admission record (AN₁) and admission orders (AO₁). If the first AN₁ and AO₁ is unacceptable, feedback information, prompt, and the original issue may be entered, and AN₁, AO₁, and the above information are submitted to the software system 40. The software system 40 then generates new AN₂ and AO₂, and this process can be repeated as needed, generating multiple sets of AN₃ and AO₃, AN₄ and AO₄, . . . , ANₓ and AOₓ. Finally, the healthcare professional selects one of these admission records and admission orders, revises the contents of the selected admission record and the selected admission order, and uses the revised admission record and the revised admission order as the final version.

The above application scenario may be modified to generate progress notes (PN), cancer treatment strategies, medical issue responses, health check-up reports, outpatient SOAP (Subjective, Objective, Assessment, Plan) analysis, nursing report analysis, various examination reports, etc. By repeatedly inputting the previous output results and feedback information into the software system 40, medical analysis reports that better meet the needs of healthcare professionals may be obtained.

In view of the above, the system and method for generating medical analysis reports proposed in the present disclosure possess advanced data analysis capabilities, excelling in interpreting complex medical records, akin to a general practitioner accurately summarizing and sequentially analyzing symptoms. Furthermore, the proposed system and method include quality control mechanisms to identify potential issues within medical reports, reducing human errors and omissions, thereby ensuring the highest quality of patient care information. Additionally, the system and method enable real-time support for medical care, automating routine medical documentation tasks, and allowing teams to share consistent and up-to-date patient records.

What is claimed is:

1. A method for generating medical analysis reports, performed by a computing device and comprising:
receiving a piece of text data;
extracting at least one piece of first sub-data from the piece of text data using a parsing module according to a natural language processing technique;
analyzing the at least one piece of first sub-data using at least one medical database and at least one rule engine to generate at least one first intermediate result;
executing a large language model to generate and output a first medical analysis report according to the at least one first intermediate result;
using the parsing module and feedback information in response to the first medical analysis report to parse the first medical analysis report or the piece of text data into at least one piece of second sub-data;
analyzing the at least one piece of second sub-data using the at least one medical database and the at least one rule engine to generate at least one second intermediate result; and
executing the large language model to generate and output a second medical analysis report according to the at least one second intermediate result.

2. The method for generating medical analysis reports of claim 1, further comprising: generating a piece of reference data using the large language model and the at least one medical database based on a retrieval-augmented generation technique, wherein the piece of reference data serves as a part of the at least one first intermediate result.

3. The method for generating medical analysis reports of claim 1, wherein analyzing the at least one piece of second sub-data using the at least one medical database and the at least one rule engine to generate the at least one second intermediate result comprises: adjusting a weight according to the feedback information to generate the at least one second intermediate result.

4. The method for generating medical analysis reports of claim 3, wherein the weight corresponds to an acceptability of the feedback information.

5. The method for generating medical analysis reports of claim 1, wherein the at least one second intermediate result is different from the at least one first intermediate result.

6. A system for generating medical analysis reports, comprising:
an input device configured to receive a piece of text data, a first medical analysis report, and feedback information;
a storage device configured to store a parsing module, at least one medical database, at least one rule engine, and a large language model;
a computing device communicably connected to the input device and the storage device, wherein the computing device is configured to: extract at least one piece of first sub-data from the piece of text data using the parsing module according to a natural language processing technique; analyze the at least one piece of first sub-data using the at least one medical database and the at least one rule engine to generate at least one first intermediate result; execute the large language model to generate and output the first medical analysis report according to the at least one first intermediate result; use the parsing module and the feedback information in response to the first medical analysis report to parse the first medical analysis report or the piece of text data into at least one piece of second sub-data; analyze the at least one piece of second sub-data using the at least one medical database and the at least one rule engine to generate at least one second intermediate result; execute the large language model to generate and output a second medical analysis report according to the at least one second intermediate result; and
an output device communicably connected to the computing device to output the first medical analysis report and the second medical analysis report.

7. The system for generating medical analysis reports of claim 6, wherein the computing device is further configured to generate a piece of reference data using the large language model and the at least one medical database based on a retrieval-augmented generation technique, wherein the piece of reference data serves as a part of the at least one first intermediate result.

8. The system for generating medical analysis reports of claim 6, wherein analyzing the at least one piece of second sub-data using the at least one medical database and the at least one rule engine to generate the at least one second intermediate result comprises: adjusting a weight used in analysis according to the feedback information to generate the at least one second intermediate result.

9. The system for generating medical analysis reports of claim 8, wherein the weight corresponds to an acceptability of the feedback information.

10. The system for generating medical analysis reports of claim 6, wherein the at least one second intermediate result is different from the at least one first intermediate result.
